(19) United States
(12) Patent Application Publication
(10) Pub. No.: US 2017/0130257 A1
(43) Pub. Date: May 11, 2017

(54) HIGH-PERFORMANCE EMPIRICAL ANALYSIS OF METABOLIC PATHWAYS IN MICROALGAE USING CELL-FREE SYSTEM

(71) Applicant: Inha-Industry Partnership Institute, Incheon (KR)

(72) Inventors: Choul Gyun LEE, Seoul (KR); Dong Myung KIM, Daejeon (KR); Seong Joo HONG, Incheon (KR)

(73) Assignee: Inha-Industry Partnership Institute, Incheon (KR)

(21) Appl. No.: 15/281,886

(22) Filed: Sep. 30, 2016

Related U.S. Application Data
(63) Continuation-in-part of application No. PCT/KR2015/003217, filed on Mar. 31, 2015.

(30) Foreign Application Priority Data
Apr. 1, 2014 (KR) 10-2014-0038634

(51) Int. Cl. C12Q 1/02

(52) U.S. Cl. CPC C12Q 1/025; G01N 2500/20

(57) ABSTRACT

For more efficient and high-performance analysis of a function of a microalgae gene, the present invention provides an empirical analysis method of a metabolic pathway using a microalgae cell-free system, including: preparing microalgae homogenate; allowing a reaction vessel to contain the microalgae homogenate, and then treating with an effector molecule; and comparing with a control, which is not treated with the effector molecule, to measure a biological effect of the effector molecule treatment.

### TECHNICAL FIELD

The present invention relates to analysis of a metabolic pathway, and more particularly to high-performance empirical analysis of a metabolic pathway in microalgae using a cell-free system.

### BACKGROUND ART

Synthetic biology is a study in which an engineering aspect is introduced into a bioscientific understanding, and includes two fields: designing and constructing a biological component and system which do not exist in the nature environment; or redesigning and constructing a biological system present in the nature environment. However, the latter meaning is more frequently used. To produce a high-performance and high efficient biological system by synthesizing genes and proteins which constitute an organism, concepts such as compartization, standardization, and modularization, which are employed in various engineering technologies, are introduced. Among these concepts, standardization of a gene is an essential process in synthetic biology which uses large amounts of genes. Standardization of a gene means construction of an information system by previously verifying transplantability of a certain genome into a certain species and performance when transplant is performed, so that time and cost can be greatly reduced, because there is no need for individual test to transplant a gene derived from other species. In addition, since a network of factors associated with certain metabolism in the body and regulatory mechanism thereof are investigated along with development of functional genomics, proteomics and metabolomics following genome project, there is an increased attempt to predict changes in the metabolic pattern due to modification, blockade, or detour of the certain metabolic pathway on the network, thereby constructing more efficient biological system.

Consequently, success and failure of synthetic biology depends on correct understanding of a function of a gene present in the body, and therefore a study to understand a correct function of a gene in a certain organism is an essential factor. Generally used methods for functional analysis of a gene include overexpression of a protein encoded by a gene by cloning the gene and then transforming the gene into a host cell, knock-out or knock-down of an inherent gene in a host cell, etc. Also, used is a method of transducing, into cells, regulatory molecules (such as a certain metabolite or inhibitor), or a nucleic acid molecule (such as an antisense oligonucleotide, or siRNA for a certain gene) to modify a metabolic pathway and then measuring the effect. For gene knock-out, a method of inducing a random mutagenesis and then selecting a loss-of-function mutant of the gene, or a method of inducing selective mutagenesis for a heterologous gene by using homologous recombination is typically used, however, in terms of efficiency, the latter is recently preferred.

However, to deliver a genetic material into cells, these typical methods should employ intracellular gene delivery methods such as transformation and transfection. In particular, for gene knock-out, efficiency is very low. Although gene knock-out is a very useful tool for prokaryotic cells having relatively simple genomes or less complicate eukaryotic cells having low copy number of a certain gene, gene knock-out is not suitable for analysis of a gene present in an organism having a complex genome or in cellular organelles such as chloroplasts and mitochondria. For microalgae, there is limitation in modeling and de novo design due to following reasons: information about base sequences of genome is insufficient; the growth rate is lower than that of bacteria; transformation efficiency is very low; genome is complicate (i.e., the size ranges from 2 Mb to 100 Mb or more, and the copy number ranges from 1 to 40) ; and a background knowledge about the genomic information is insufficient.

Although techniques have been developed to stably deliver a heterologous gene into a chromosome in microalgae (see U.S. Pat. No. 5,661,017, U.S. Pat. No. 8,119,859, etc.), there is a problem in that these methods are specifically applied to a limited species, or the inserted heterologous gene is missing after continuous passage, because inserting the heterologous gene into the same location of every chromosome is practically unavailable in microalgae having a high copy number. Moreover, for transformation of microalgae, only one heterologous gene can be introduced into the microalgae at one time, so that a study about an effect obtained by introducing a plurality of genes is difficult.

Thus, a novel approach is required which is widely applied to various species of microalgae and capable of empirically analyzing a function of a gene in microalgae and a metabolic pathway associated with the gene in a high-throughput way.

### DISCLOSURE OF THE INVENTION

### Technical Problem

The present invention is to solve various problems including the above-described problem. An object of the present invention is to provide a high-performance analysis method for a function of a gene in microalgae. However, the problem is illustrative, and the scope of the present invention is not limited thereto.

### Technical Solution

According to one aspect of the present invention,

provided is an empirical analysis method of a metabolic pathway using a microalgae cell-free system, including:

preparing a microalgae homogenate;

allowing a reaction vessel to contain the microalgae homogenate, and then treating with an effector molecule; and

comparing with a control, which is not treated with the effector molecule, to measure a biological effect of the effector molecule treatment.

According to another aspect of the present invention,

provided is a high-performance empirical analysis method of a metabolic pathway using a microalgae cell-free system, including:

preparing a microalgae homogenate;

allowing a multiwall reaction vessel to contain the microalgae homogenate, and then treating with an effector molecule library;

measuring a biological effect according to the effector molecule treatment; and

comparing with a control, which is not treated with the effector molecule, and selecting an effector molecule which significantly alters the biological effect.

According to still other aspect of the present invention, provided is a kit for high-performance empirical analysis of a metabolic pathway of microalgae including microalgae homogenate.

### Advantageous Effects

According to one embodiment of the present invention as described above, rapid analysis of a microalgae gene function and more efficient high-throughput analysis for a relating metabolic pathway are achieved by simply adding the effector molecule into microalgae homogenate without complex and inefficient genetic manipulation (such as gene delivery into microalgae cells or knock-out of a gene inherent in the genome), thereby largely contributing to microalgae synthetic biology. Surely, the scope of the present invention is not limited to the effect.

### BRIEF DESCRIPTION OF THE DRAWINGS

FIG. 1 is a schematic diagram schematically showing a kit for analyzing a microalgae gene according to an embodiment of the present invention.

FIG. 2 is a schematic diagram schematically showing processes of a method for analyzing a microalgae gene or a method for screening an effector molecule according to an embodiment of the present invention.

FIG. 3 is a series of graphs showing concentration of chlorophyll a (A) and concentration of protein extracts using sonication and French press.

FIG. 4 is a schematic diagram schematically showing a metabolic pathway of phycoerythrobilin according to to an embodiment of the present invention.

FIG. 5 is a series of optical absorbance spectrums improving synthesis of biliverdin IXa and phycoerythrobilin synthesized from 5-ala (A) and HEME (B), respectively. Black arrow indicates biliverdin IXa having absorption spectra of 440 and 660 nm and white arrow indicates phycoerythrobilin having an absorption spectrum of 550 nm.

FIG. 6 is a schematic diagram schematically showing a metabolic pathway of astaxanthin biosynthesis which is analyzable according to an embodiment of the present invention.

FIG. 7 is a schematic diagram schematically showing a terpenoid biosynthesis pathway which is a precursor pathway of astaxanthin biosynthesis analyzable according to an embodiment of the present invention.

FIG. 8 is a schematic diagram schematically showing a metabolic pathway of fatty acid methyl ester (FAME) biosynthesis according to an embodiment of the present invention.

### MODE FOR CARRYING OUT THE INVENTION

Definition:

The term “homogenate” used herein means a suspension obtained by lysing cells, and may include particular intact cellular organelles such as mitochondria, cell nucleus, and chloroplast as well as lysate of the cellular organelles.

The term “effector molecule” used herein means a subject molecule for a test, wherein the molecule is expected to induce a certain biological effect, when the molecule is added to cell homogenate. The molecule may be a gene construct, which may be expressed to a protein when being added to cell homogenate via a transcriptional and translational factors in the cell homogenate, a nucleic acid molecule such as an antisense oligonucleotide, siRNA, shRNA, miRNA, clustered regularly interspaced short panlindromic repeats (CRISPRs) nucleotide, or transcription activator-like effector nuclease (TALEN) nucleotide, which may inhibit expression of a certain gene, an antibody, which inhibits a function of a certain protein, or a functional fragment thereof, an aptamer, a small compound inhibitor, or, when the certain protein is an enzyme, a substrate analogue which has a structure similar to that of the substrate of the enzyme, but may competitively or noncompetitively inhibit the function of the enzyme. Also, the effector molecule may be a gene derived from metagenome. The term effector molecule may be replaced by the term effector molecule candidate with regard to that the biological effect of the molecule is not investigated yet.

The term “effector molecule library” used herein means two or more same lineage of effector molecules, or a group of effector molecule candidates. The library includes a genomic library, a cDNA library, an aptamer library, an antibody library, a small compound library, a phage display library, etc. By individually applying effector molecule candidates belonging to these libraries to the screening method according to an embodiment of the present invention, an effector molecule candidate having a desired property can be selected.

The term “CRISPR′” used herein is an abbreviation of the term “clustered regulatory interspaced short palindromic repeat”, and means a DNA locus including short repeat of a nucleic acid sequence. The CRIPSR/Cas system for gene editing has developed in 2013 by Ran et al. (see Ran et al., Cell 154(6): 1380-1389, 2013). By using the CRIPSR/Cas system, a desired position on genomic DNA in the body may be cleaved by delivering Cas9 protein and appropriate guide DNA.

### DETAILED DESCRIPTION OF THE INVENTION:

Hereinafter, the present invention will be described in more detail:

According to one aspect of the present invention,

provided is an empirical analysis method of a metabolic pathway using a microalgae cell-free system, including:

preparing a microalgae homogenate;

allowing a reaction vessel to contain the microalgae homogenate, and then treating with an effector molecule; and

comparing with a control, which is not treated with the effector molecule, to measure a biological effect of the effector molecule treatment.

In the method, the microalgae may belong to Nostoc sp., Anabaena sp., Crocosphaera sp., Cyanothece sp., Trichormus sp., Richella sp. Calothrix sp., Botryococcus sp., Chlorella sp., Crypthecodinium sp., Arthrospira sp., Cylindrotheca sp., Dunaliella sp., Isochrysis sp., Monallanthus sp., Nannochloris sp., Nannochloropsis sp., Neochloris sp., Nitzschia sp., Phaeodactylum sp., Schizochytrium sp., Tetraselmis sp., or Haematococcus sp.

In the method, the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase. Additionally, the microalgae may be a lysate including cellular organelles such as nucleus, mitochondria, and chloroplast. All or a part of these components may be retained, or all or a part of these components may be externally added depending on the purpose. Since the microalgae homogenate may reproduce transcription and translation of a gene, and posttranslational modification as well as metabolic pathways through various enzymes, the microalgae homogenate may be conveniently used to construct a regulatory model of a function of an effector molecule such as a certain gene or inhibitor in cells and a metabolic pathway through the molecule without complex and inefficient genetic manipulation such as transformation, transfection, or homologous recombination. In particular, the microalgae homogenate may include intact chloroplast for a study about photosynthesis efficiency. It has been well known that the cellular organelles may be separated through methods including centrifugation or ultracentrifugation due to differences in specific gravity (Michelsen et al., Methods Enzymol., 463: 305-328, 2009; Dashek, W. V., Methods in Plant Microscopy and Cytochemistry, 161-167, 2000).

In the method, the effector molecule may be a gene construct in which a polynucleotide encoding a certain protein is operatively linked to a promoter operated in microalgae, siRNA, shRNA, miRNA, clustered regularly interspaced short panlindromic repeats (CRISPRs, Horvath and Barrangou, Science, 327(5962): 167-170, 2010) nucleotide, transcription activator-like effector nuclease (TALEN, Boch, Nat. Biotech., 29(2):135-136, 2011) nucleotide, or an antisense nucleotide, which may inhibit expression or function of a certain gene inherent in microalgae, an antibody, which inhibits a function of an intracellular protein in microalgae, or a functional fragment thereof, or a small compound inhibitor, a substrate of an intracellular enzyme in microalgae, or a substrate analogue which acts as an inhibitor.

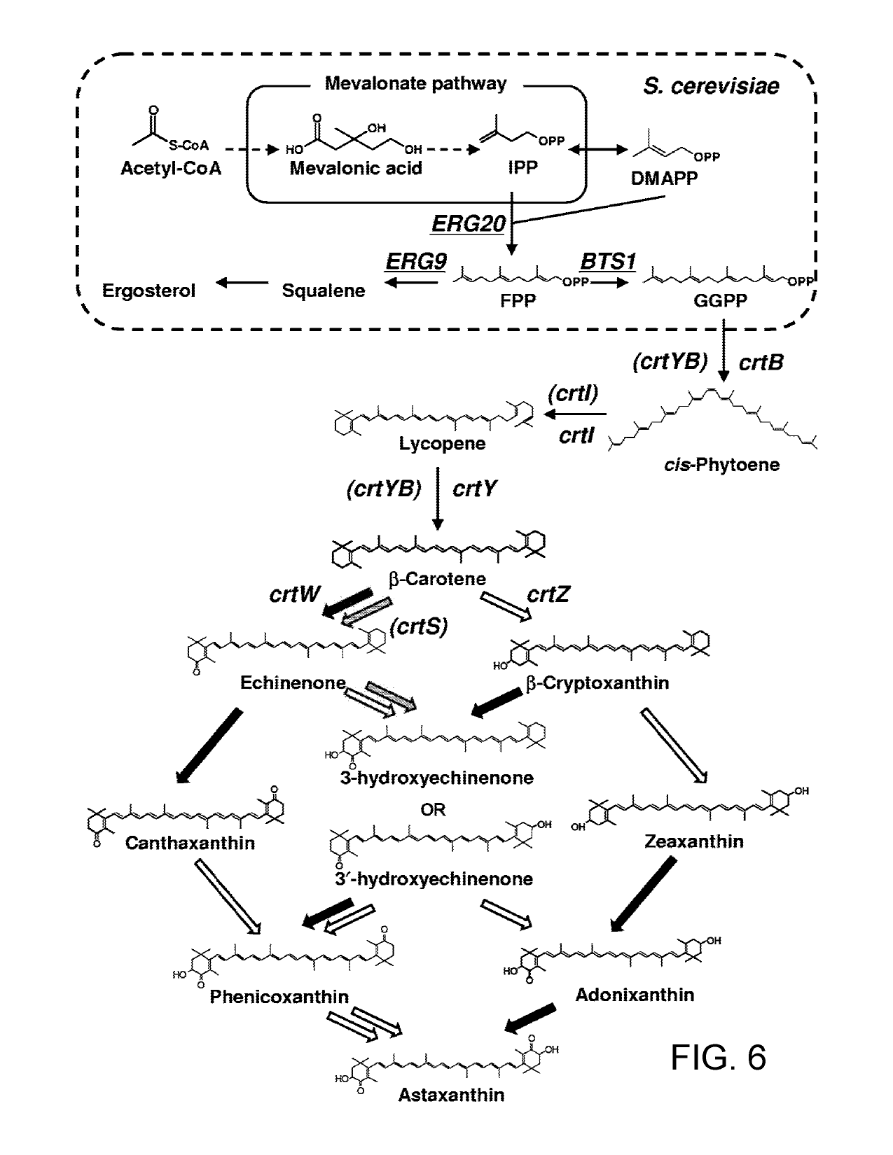

In the method, the biological effect may be appropriately set according to the property of the treated effector molecule. For example, when the metabolic pathway is astaxanthin biosynthesis pathway and the treated effector molecule is a gene construct including a polynucleotide encoding an enzyme associated with biosynthesis of a pigment such as astaxanthin (see FIG. 6), the biological effect may be a change in an amount of astaxanthin production. The change in the amount of astaxanthin production may be evaluated by measuring absorbance at a certain wavelength of astaxanthin (e.g., 478.8 nm for (3S, 3′S)13-trans-astaxanthin having a useful biological activity). When the treated effector molecule is a candidate for regulating an enzyme associated with lipid synthesis, the biological effect may be a change in an amount of liquid production. In this case, the amount of lipid production may be measured by using fluorescence detection reported by Listenberger et al. (see Listenberger et al., Curr. Protoc. Cell Biol., 2007, Chapter 24, Unit 24.2.). When the microalgae is a hydrogen-producing strain, and a metabolic pathway to be analyzed is a metabolic pathway associated with hydrogen production, the biological effect may be a change in an amount of hydrogen production, and the change in the amount of hydrogen production may be evaluated through various hydrogen detecting sensors (Li et al., Nanoscale Res. Lett., 9(1): 118, 2014; Al-Hinai et al., Faraday Discuss., 164: 71-91, 2013). When a metabolic pathway to be analyzed is a metabolic pathway associated with ATP production, the biological effect may be a change in an amount of ATP production, and the change in the amount of ATP production may be evaluated by treating the reaction vessel with luciferin and luciferase and measuring bioluminescence through a luminometer.

In the method, the reaction vessel may be a single test tube, or 6-well, 12-well, 24-well, 48-well, 96-well, 192-well, or 384-well microplate. For a microplate, an experiment may be performed in a high-throughput way by using various genes as a target at the same time. Moreover, an experiment may be performed in a more rapid and high-throughput way by using a microarray or a microfluidic reaction chamber. The method according to an embodiment of the present invention has an advantage in that the biological effect may be immediately evaluated by using a detector such as a UV-VIS spectrophotometer, a flourometer, or a luminometer simultaneously or after performing the reaction in the reaction vessel as described above.

According to another aspect of the present invention,

provided is a high-performance empirical analysis method of a metabolic pathway using a microalgae cell-free system, including:

preparing a microalgae homogenate;

allowing a multiwall reaction vessel to contain the microalgae homogenate, and then treating with an effector molecule library;

measuring a biological effect according to the effector molecule treatment; and

comparing with a control, which is not treated with the effector molecule, and selecting an effector molecule which significantly alters the biological effect.

In the screening method, the microalgae may belong to Nostoc sp., Anabaena sp., Crocosphaera sp., Cyanothece sp., Trichormus sp., Richella sp., Calothrix sp., Botryococcus sp., Chlorella sp., Crypthecodinium sp., Arthrospira sp., Cylindrotheca sp., Dunaliella sp., Isochrysis sp., Monallanthus sp., Nannochloris sp., Nannochloropsis sp., Neochloris sp., Nitzschia sp., Phaeodactylum sp., Schizochytrium sp., Tetraselmis sp., or Haematococcus sp.

In the screening method, the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase. Additionally, the microalgae may be a lysate including cellular organelles such as nucleus, mitochondria, and chloroplast. All or a part of these components may be retained, or all or a part of these components may be externally added depending on the purpose. In particular, the microalgae homogenate may include intact chloroplast for a study about photosynthesis efficiency.

In the screening method, the effector molecule library may be a gene construct library in which a polynucleotide encoding a certain protein is operatively linked to a promoter operated in microalgae, a gene construct having a promoter, which is operated by an externally added RNA polymerase, an mRNA library prepared through external transcription, a library of siRNA, shRNA, miRNA, antisense nucleotide, clustered regularly interspaced short panlindromic repeats (CRISPRs, Horvath and Barrangou, Science, 327(5962): 167-170, 2010) nucleotide, or transcription activator-like effector nuclease, (TALEN, Boch, Nat. Biotech., 29(2):135-136, 2011) nucleotide, which may inhibit expression or functions of certain genes inherent in microalgae, a library of an antibody, which inhibits a function of an intracellular protein in microalgae, or a functional fragment thereof, or a small compound inhibitor, a library of substrates of an intracellular enzyme of microalgae, or a substrate analogue library which acts as an inhibitor. Also, the effector molecule library may be random mutant or site-directed mutant library of a gene having a known nucleic acid sequence, or an expressed sequence tag (EST) library cloned from metagenome. In particular, the EST library derived from metagenome may employ various genetic information of microalgae, which is difficult to culture or may not be cultured, so that the EST library may contribute to preparation of transformed microalgae having a desired property as well as understanding of a certain metabolic pathway and development of microalgae synthetic biology, when being applied to the screening method according to an embodiment of the present invention.

In the screening method, the biological effect may be appropriately set according to the property of the treated effector molecule library. For example, the treated effector molecule library is a gene construct library including a polynucleotide encoding an enzyme associated with biosynthesis of a pigment such as astaxanthin, the biological effect may be a change in an amount of astaxanthin production. The change in the amount of astaxanthin production may be evaluated by measuring absorbance at a certain wavelength of astaxanthin (e.g., 478.8 nm for (3S, 3′S)13-trans-astaxanthin having a useful biological activity). When the treated effector molecule library is a candidate for regulating an enzyme associated with lipid synthesis, the biological effect may be a change in an amount of liquid production. In this case, the amount of lipid production may be measured by using fluorescence detection reported by Listenberger et al. (see Listenberger et al., Curr. Protoc. Cell Biol., 2007, Chapter 24, Unit 24.2.). When the microalgae is a hydrogen-producing strain, and a gene to be analyzed is a gene associated with hydrogen production, the biological effect may be a change in an amount of hydrogen production, and the change in the amount of hydrogen production may be evaluated through various hydrogen detecting sensors (Li et al., Nanoscale Res. Lett., 9(1): 118, 2014; Al-Hinai et al., Faraday Discuss., 164: 71-91, 2013). When a metabolic pathway to be analyzed is a metabolic pathway associated with ATP production, the biological effect may be a change in an amount of ATP production, and the change in the amount of ATP production may be evaluated by treating the reaction vessel with luciferin and luciferase, and then measuring bioluminescence through a luminometer.

In the screening method, the reaction vessel may be a single test tube, or 6-well, 12-well, 24-well, 48-well, 96-well, 192-well, or 384-well microplate. For a microplate, an experiment may be performed in a high-throughput way by using various genes as a target at the same time. Moreover, an experiment may be performed in a more rapid and high-throughput way by using a microarray or a microfluidic reaction chamber. The method according to an embodiment of the present invention has an advantage in that the biological effect may be immediately evaluated by using a detector such as a UV-VIS spectrophotometer, a flourometer, or a luminometer simultaneously or after performing the reaction in the reaction vessel as described above.

According to another aspect of the present invention, provided is a kit for analyzing a function of a microalgae gene including microalgae homogenate. In the kit, the microalgae may belong to Nostoc sp.,

Anabaena sp., Crocosphaera sp., Cyanothece sp., Trichormus sp., Richella sp. Calothrix sp., Botryococcus sp., Chlorella sp., Crypthecodinium sp., Arthrospira sp., Cylindrotheca sp., Dunaliella sp., Isochrysis sp., Monallanthus sp., Nannochloris sp., Nannochloropsis sp., Neochloris sp., Nitzschia sp., Phaeodactylum sp., Schizochytrium sp., Tetraselmis sp., or Haematococcus sp.

In the kit, the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase. Additionally, the microalgae may be a lysate including cellular organelles such as nucleus, mitochondria, and chloroplast. All or a part of these components may be retained, or all or a part of these components may be externally added depending on the purpose.

The kit may further include an effector molecule library.

The effector molecule library may be a gene construct library in which a polynucleotide encoding a certain protein is operatively linked to a promoter operated in microalgae, a library of a siRNA, shRNA, miRNA, antisense nucleotide, clustered regularly interspaced short panlindromic repeats (CRISPRs, Horvath and Barrangou, Science, 327(5962): 167-170, 2010) nucleotide, or transcription activator-like effector nuclease, (TALEN, Boch, Nat. Biotech., 29(2):135-136, 2011) nucleotide, which may inhibit expression or functions of a certain gene inherent in microalgae, a library of an antibody, which inhibits a function of an intracellular protein in microalgae, or a functional fragment thereof, or a small compound inhibitor, a library of substrates of an intracellular enzyme of microalgae, or a substrate analogue library which acts as an inhibitor. Also, the effector molecule library may be random mutant or site-directed mutant library of a gene having a known nucleic acid sequence, or an expressed sequence tag (EST) library cloned from metagenome.

Hereinafter, examples of the present invention will be described in more detail with reference to the accompanying drawings. The present invention, however, be embodied in different forms and should not be construed as limited to the examples set forth herein. Rather, these examples are provided so that this disclosure will be thorough and compete, and will fully convey the scope of the invention to those skilled in the art. In the drawings, the dimensions are exaggerated and shrink for clarity of illustration.

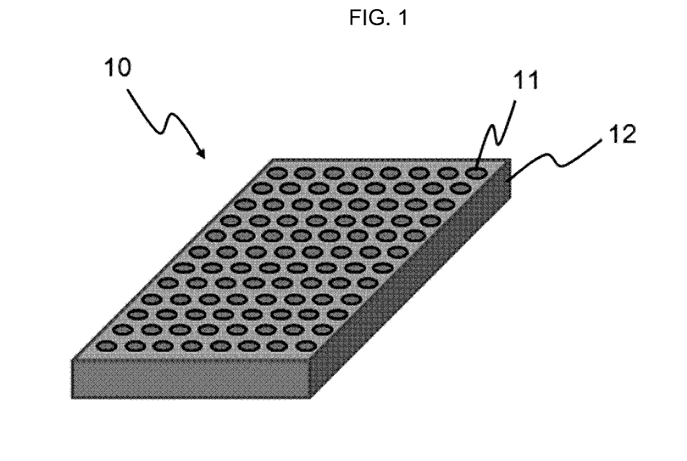

FIG. 1 is a schematic diagram schematically showing a kit 10 for analyzing a microalgae gene according to an embodiment of the present invention. The kit containing microalgae homogenate 11 in a reaction vessel 12 may be provided, or a separately provided microalgae homogenate 11 may be dispensed into the reaction vessel 12 immediately before use in an appropriate amount by a user as needed.

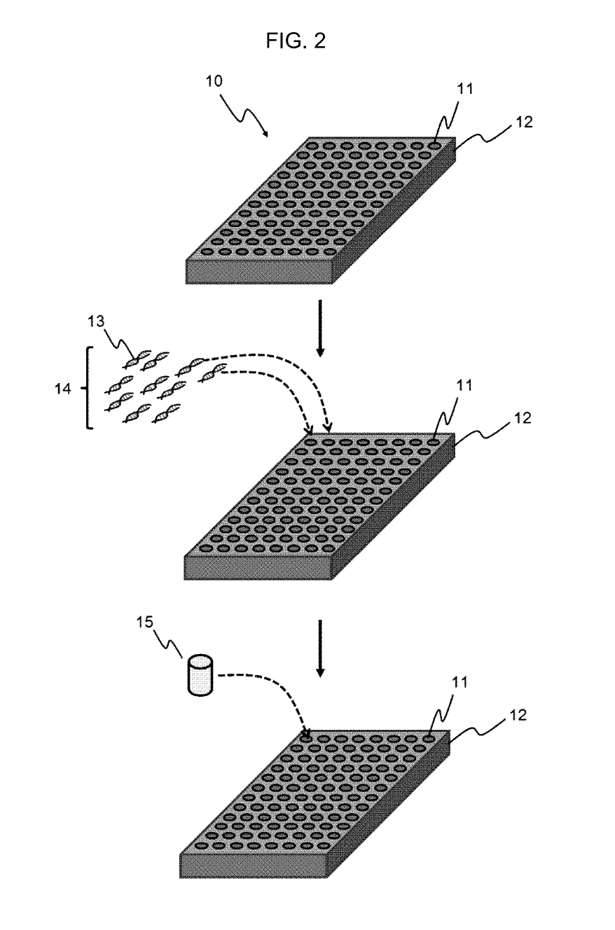

FIG. 2 is a schematic diagram schematically showing processes of a method for analyzing a microalgae gene or a method for screening an effector molecule according to an embodiment of the present invention. Microalgae homogenate 11 is dispensed in the reaction vessel 12, which is a 96-well microplate. Then, an effector molecule 13 to be analyzed or each effector molecule 13 in an effector molecule library 14 is added to each well of the reaction vessel 12 to perform the metabolic reaction. Thereafter, a reaction reagent 15 is treated to evaluate a biological change as needed, and then the result of reaction due to the reagent is detected by using various measuring instruments or sensors. When the reactant itself is a material showing fluorescence or having absorbance at a certain wavelength, a degree of production of the reactant may be quantified immediately after completion of the reaction without additional treatment with the reagent 15.

Hereinafter, the present invention will be described in more detail with reference to the examples. However, these examples are provided so that this disclosure will be thorough and compete, and will fully convey the scope of the invention to those skilled in the art. The scope of the present invention is not limited to the examples.

### EXAMPLE 1

### Preparation of Algal Homogenate

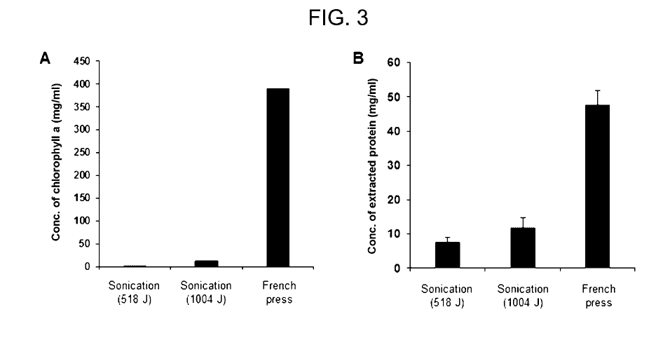

The present inventors applied two methods (sonication and French press) in order to prepare homogenate of microalgae (Synechocystis sp. pcc 6803). For the sonication, 518 and 1004 J of energy using 52 W power supply, respectively. All samples were positioned in an ice bucket as being sonicated in order to prevent loss of function of proteins due to heat and sonication for 10 sec and cooling for 50 sec were repeated. In addition, for the French press, 1,300 psi of pressure was used for disrupting the microalgae. In order to compare the two methods, contents of chlorophyll a and protein were detected. As a result, French press showed 388.8 mg/ml of chlorophyll which is 32 times higher than that of sonication and 47.7 mg/ml of protein which is 4 times higher than that of sonication (FIG. 3). From the result, it is recognized that the two methods can be used for disrupting cells in preparing cell homogenate and French press is the best method for disrupting algal cells.

### EXAMPLE 2

### Synthesis of Phycoerythrobilin Using Cell-Free System

Microalgal pigments such as carotene, xanthophyll, lutein, chlorophyll, phycobilin and phycobiliprotein are acknowledged their industrial applicability as functional materials. They have been used as medicinal materials due to their anti-cancer, anti-aging, anti-HIV and anti-inflammatory activities or as food additives such as natural pigments being added to cadies, ice creams, dairy products and soft drinks. It has been tried to synthesis phycobilin or phycobiliprotein by introducing genes encoding enzymes involved in synthesizing pigments into E. coli. However, one has to optimize recombinant E. coli for a long time considering factors related to transcription and translation of various genes in order to optimize the pigment synthesis. On the contrary cell-free synthesis system can be used to synthesis target pigments by preparing enzymes related to pigment synthesis in an independent reactor and then reacting the enzymes with needed substrates in a single reactor. Thus from this approach using cell-free synthesis system, one can obtain information such as optimal concentration of co-enzyme and optimal ratio of enzymes involved in the pigment synthesis in a short time. One can produce pigments with a maximum yield in the optimal condition by scaling up based on the information.

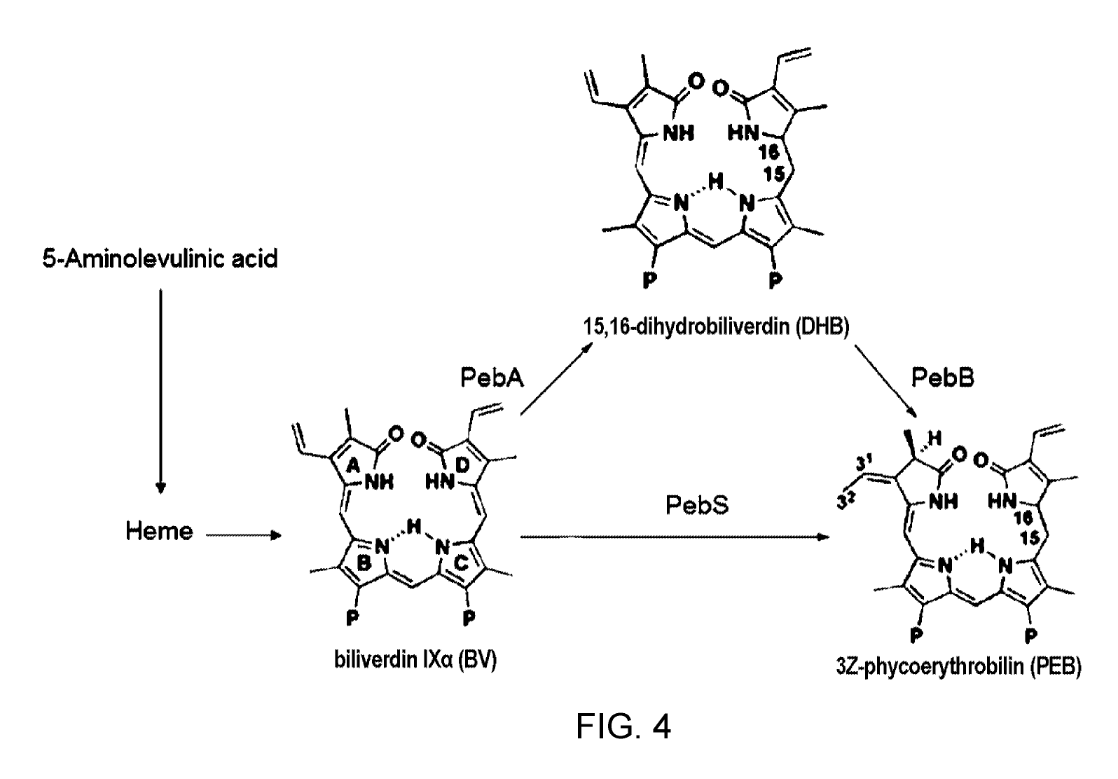

In the example, the present inventors founded a cell-free synthesis system for synthesizing phycoerythrobilin. Biosynthetic pathway of phycoerythrobilin is shown as FIG. 4. E. coli-based cell-free protein synthesis system has a metabolic pathway for synthesizing HEME and gene encoding HEME oygenase (Hol) derived from Synechocystis sp. 6803, 15,16-dihydrobiliverdin:ferreddoxin oxidoreductase (PebA), phycoerythrobilin:ferredoxin oxidoreductase (PebB) which were derived from Sinecococcus sp. and phycoerythrobilin synthase (PebS) derived from Prochlorococcus phage P-SSM2 was synthesized by gene synthesis based on genetic information in order to obtain enzymes involved in the phycoerythrobilin synthesis. The synthesized genes were inserted to plasmids for in vitro transcription and translation using T7 promoter/terminator. Hol was used to convert HEME to biliverdin IXa and two pathways including PebA-PebB pathway and PebS pathway were used to synthesize phycoerythrobilin from the biliverdin IXa in order to synthesize the phycoerythrobilin.

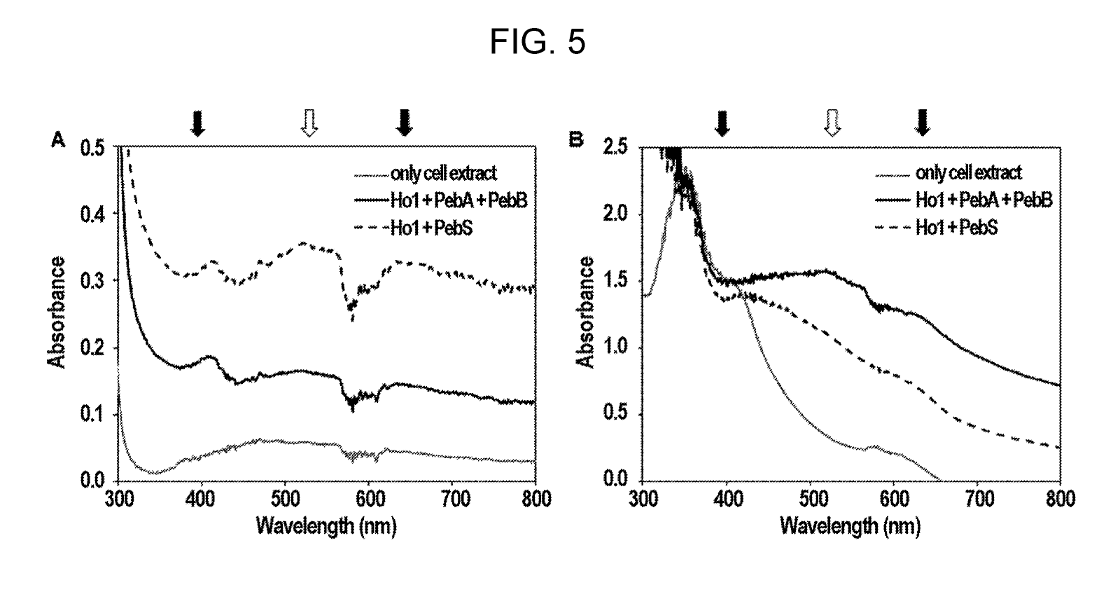

The relevant enzymes ware synthesized in each individual reaction chamber using the cell-free protein synthesis system and enzymes related to one reaction chamber were mixed without isolation or purification and 5-aminolevulinic acid (5-ala) and hemin as start materials for the phycoerythrobilin synthesis were supplied in order to perform a reaction for synthesizing the pycoerythrobilin. After the synthetic reaction for 6 hours at 30° C. biliverdin IXa which is an intermediate and phycoerythrobilin which is a target pigment were analyzed by using optical absorbance. As a result, the biliverdin IXa was detected by observing optical absorbance at 440 nm and 660 nm and the phycoerythrobilin was detected when observing optical absorbance at 550 nm (FIG. 5). From the results, it is confirmed that the intermediate and the target pigment which is a final product are coexist within the reaction solution.

### EXAMPLE 3

### Investigation of Astazanthin Producing Enzyme Library

The astazanthin biosynthesis metabolic pathway is shown in FIG. 6. FPP is converted into lycopene by crtE and crtB. Then, lycopene is converted into beta-carotene by crtY, and beta-carotene is finally converted into astazanthin via zeaxanthin by crtZ and crtW. For eukaryotes, genes associated with beta-carotene and astazanthin biosynthesis as described above are regulated in an operon unit. Thus, microorganisms, which exist in the environment, but are not isolated and identified, are highly likely to include a gene for astazanthin biosynthesis. Therefore, a gene library may be constructed from metagenome by using a high conservative region among genes which are associated with astazanthin biosynthesis and have a typically known nucleic acid sequence. To investigate a gene capable of increasing astazanthin production yield, when being introduced into microalgae among genes belonging to metagenome gene library constructed as above, the screening method according to an embodiment of the present invention may be used.

Specifically, astazanthin-related metagenome is extracted from a sample taken from sea water, and 100 ng of library DNA constructed in a plasmid, phosmid, or BAC vector is prepared.

Astazanthin biosynthesis microalgae, i.e., a Haematococcus pluvialis strain is cultured under a sterile condition. Cells are collected through a centrifuge and resuspended in a buffer including magnesium acetate, and potassium acetate. Then cells are washed through a centrifuging process. This process is repeated three times.

The washed cells are collected through centrifugation, and cells are disrupted by using a french press. The cell lysate is subjected to centrifugation to prepare cell homogenate in which precipitates are removed. 40 ul of the cell homogenate is homogenously introduced into two 96-well plates containing the library prepared above (control and experimental group). Then, 60 ul of a reaction solution {HEPES-KOH (final concentration 30 mM, pH 7.6, Merck, USA), Mg(OAc)₂ (final concentration 2.5 mM), KOAc (final concentration 75 mM), amino acid (final concentration 100 μM), spermidine (final concentration 0.25 mM) and energy reproducing elements (final concentration 1.75 mM ATP, final concentration 0.3 mM GTP, final concentration 0.3 mM CTP, final concentration 0.3 mM UTP)} for a cell-free synthesis system is added(Stech et al., PLoS One, 9(5): e96635, 2014). For the control, a precursor for astazanthin synthesis (e.g., glyceraldehyde 3-phosphatge, pyruvate, acetyl CoA and mevalonate) (FIG. 6), and an energy source of an organic carbon source (a final concentration of 33 mM of glucose, glucose-6-phosphate, fructose-1,6-bisphosphate, 3-phosphoglycerate, phosphophenol pyruvate or sodium pyruvate) are added. For the experimental group, high intensity (1,000 μEmol/m²/s) light and 2% carbon dioxide (or 1 g/L of sodium bicarbonate), which is an inorganic carbon source, are added and the resultants are allowed to react at 25° C. for 4 hours. After the reaction is terminated, absorbance at 478.8 nm is measured on a UV-VIS spectrophotometer. The absorbance is relative absorbance obtained by subtracting absorbance of the control in which the DNA library is not added. The astazanthin concentration is quantified by using a standard solution in which astazanthin is serially diluted in homogenate of a mutant Haematococcus pluvialis strain whose ability to produce astazanthin is removed.

### EXAMPLE 4

### Investigation of Gene Associated with Lipid Production

When biodiesel is prepared by using various fatty acids produced by microalgae, cold flow, oxidation stability, as well as energy density of the prepared biodiesel are varied according to the length or saturation degree of fatty acids. Accordingly, when these fatty acids are produced by using the same quantity of energy source, an optimal fatty acid may vary depending on places or seasons. Required is a method to increase beneficial fatty acid content through selection of microalgae, which accumulates a lipid including the optimal fatty acid as a main fatty acid in cells in a large amount, or by artificially modifying the typical lipid metabolism of microalgae through genetic manipulation. The gene analysis method according to an embodiment of the present invention may be useful to investigate that enhancement of expression of which gene and inhibition expression of which gene, among various genes associated with lipid metabolism, lead to increase in beneficial fatty acid content and absolute amount of lipid production.

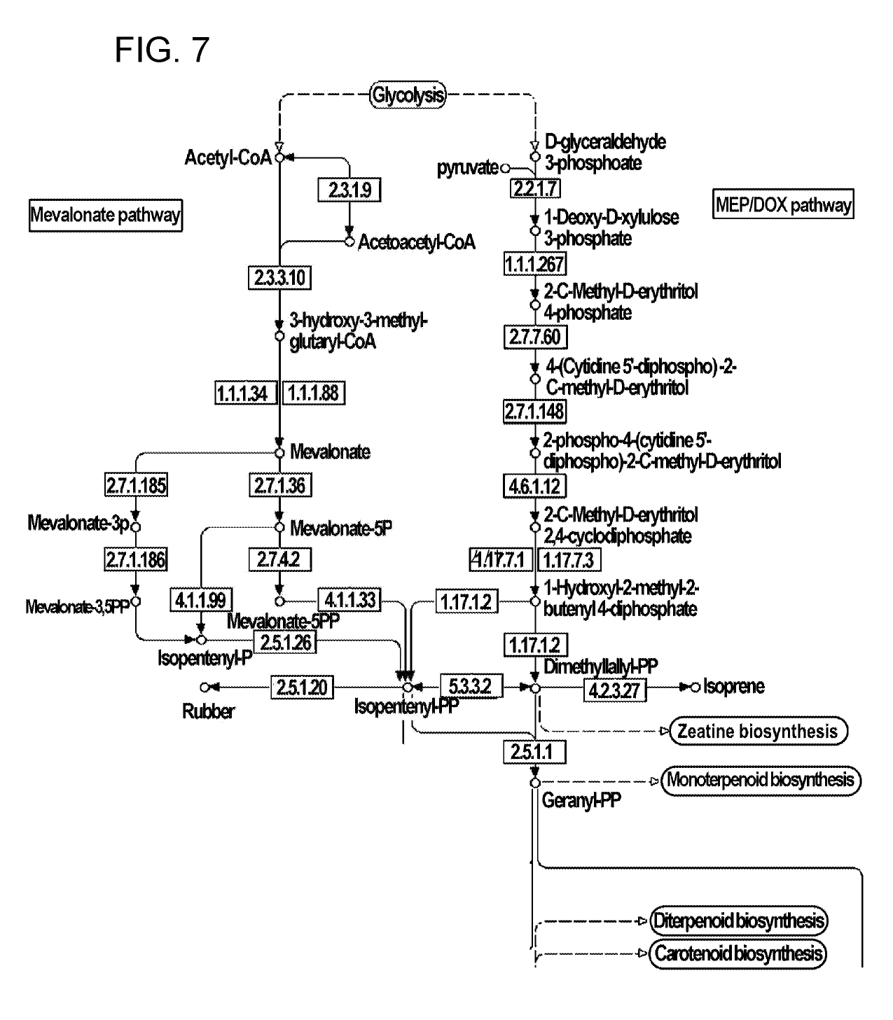

Specifically, an siRNA library capable of inhibiting a function of a gene associated with lipid metabolism is constructed. Then, a Chlamydomonas reinhardtii strain is cultured under a sterile condition. Cells are collected through a centrifuge and resuspended in a buffer including magnesium acetate, and potassium acetate. Then cells are washed through a centrifuging process. This process is repeated three times. Thereafter, the washed cells are collected through centrifugation, and cells are disrupted by using a french press. The lysate is subjected to centrifugation to prepare cell homogenate in which precipitates are removed. 40 ul of the cell homogenate and ul of the reaction solution for a cell-free synthesis system used in Example 1 are homogenously introduced into two 96-well plates containing the siRNA library constructed above (control and experimental group). For the control, a precursor for lipid synthesis (e.g., glyceraldehyde 3-phosphatge, pyruvate, acetyl CoA and mevalonate) (FIG. 7), and an energy source of an organic carbon source (a final concentration of 33 mM of glucose, glucose-6-phosphate, fructose-1,6-bisphosphate, 3-phosphoglycerate, phosphophenol pyruvate or sodium pyruvate) are added. For the experimental group, 40 μEmol/m²/s of light and 2% carbon dioxide (or 1 g/L of sodium bicarbonate), which is an inorganic carbon source, are added and the resultants are allowed to react at 25° C. for 4 hours (FIG. 7). A fluorescent reagent (Bodipy or Nile red), which reacts to lipid, is added to the reaction solution, and allowed to react at room temperature for 10 minutes. A relative value is quantified by comparing florescent intensity with that of the control which does not contain the siRNA by using a flourometer (Bodipy: excitation wavelength 488 nm, emission wavelength 515 nm; Nile red: excitation wavelength 530 nm, emission wavelength 575 nm). The quantification is performed through microassay using GC-MS.

### EXAMPLE 5

### Investigation of Gene Associated with Photosynthesis Efficiency Improvement

Microalgae produces bioenergy by using light-dependent reaction of photosynthesis, and synthesizes an organic carbon source C3 compound from CO₂, which is an inorganic carbon source, through light-independent reaction of photosynthesis. Accordingly, when photosynthesis efficiency is improved, synthesis of the organic carbon source from the inorganic carbon source is increased. Thus, the growth of microalgae may be enhanced or synthesis of beneficial materials may be increased. Required is a method to increase photosynthesis efficiency by artificially modifying redesign of photosynthesis-related metabolic pathway or enzyme optimization through genetic manipulation. The empirical analysis method of a metabolic pathway according to an embodiment of the present invention may be useful to investigate that enhancement of expression of which gene and inhibition of expression of which gene lead to increase in microalgae biomass and absolute photosynthesis efficiency through insertion and deletion of various genes associated with lipid metabolism.

Specifically, a clustered regularly interspaced short palindromic repeats (CRISPR) library capable of inhibiting a function of a gene associated with photosynthesis is constructed (Ran et al., Cell 154(6): 1380-1389, 2013). Sequentially, a Dunaliella tertiolecta strain is cultured under a sterile condition. Cells are collected through a centrifuge, resuspended in a buffer including magnesium acetate, and potassium acetate, and washed through a centrifuging process. This process is repeated three times. Thereafter, the washed cells are collected through centrifugation, and cells are disrupted by using a french press. The lysate is subjected to centrifugation to prepare cell homogenate in which precipitates are removed. 40 ul of the cell homogenate followed by 60 ul of the reaction solution for a cell-free synthesis system used in Example 1 is homogenously introduced into two 96-well plates containing the CRISPR plasmid library (control and experimental group). Then, 40 μEmol/m²/s of light and carbon dioxide (or 1 g/L of sodium bicarbonate), which is an inorganic carbon source, are added and the resultants are allowed to react at 25° C. for 4 hours. Thereafter, fluorescent intensity (excitation wavelength 460 nm, emission wavelength 698 nm-708nm or 730 nm-740 nm) of chloroplast in the reaction solution is measured through a fluorometer.

### EXAMPLE 6

### Production of Fatty Acid Methyl Ester (Fame) Using Microalgae and Comparison of Productivity According to Photoautotroph/Heterotrophy Condition

The microalgae cell-free system according to an embodiment of the present invention may be used in a study about improvement of productivity of biodiesel production depending on various conditions or regulation of genes.

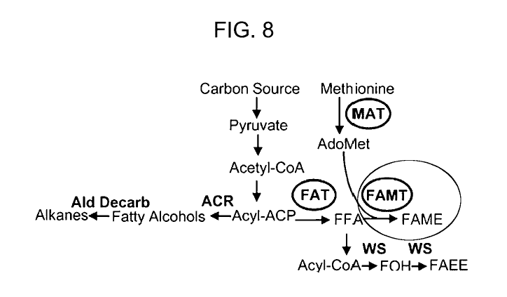

Specifically, a Synechocystis sp. strain is transformed with a plasmid including fatty acid methyltransferase, (FAMT) (FIG. 8) gene, which is derived from M. marinum and M. smegmatis capable of producing methyl ester from free fatty acids, and inserted into a genome of Synechocystis sp. through homologous recombination (Nawabi et al., Appl. Environ. Microbiol., 77(22): 80528061, 2011). The transformed Synechocystis sp. strain is cultured. Cells are collected through a centrifuge and resuspended in a buffer including magnesium acetate, and potassium acetate. Then, cells are washed through a centrifuging process. This process is repeated three times. Thereafter, the washed cells are collected through centrifugation, and cells are disrupted by using a french press. The lysate is subjected to centrifugation to prepare cell homogenate in which precipitates are removed. 15 ul of a reaction solution [1.2 mM ATP, GTP, UTP, and CTP (0.85 mM for each), 34.0 μg/mL L-5-formyl-5,6,7,8-tetrahydrofolic acid (folinic acid), 170.0 μg/mL tRNA mixture, 130 mM potassium glutamate, 10 mM ammonium glutamate, 12 mM magnesium glutamate, 20 amino acids (2 mM for each), 0.33 mM nicotinamide adenine dinucleotide, NAD), 0.27 mM coenzyme-A (Co-A), 1.5 mM spermidine, 1 mM putrescine, 4 mM sodium oxalate, 100 μg/mL T7 RNA polymerase, energy reproducing elements {phosphoenolpyruvate, polyphosphate, creatine-phosphate, etc.} and 27% v/v cell homogenate] for cell-free synthesis system including cell homogenate is added to two RNase-free centrifugation tubes (control and experimental group). For the control, a precursor for astazanthin synthesis and an energy source of an organic carbon source (a final concentration of 33 mM of glucose, glucose-6-phosphate, fructose-1,6-bisphosphate, 3-phosphoglycerate, phosphophenol pyruvate or sodium pyruvate) are added. For the experimental group, 20 μEmol/m²/s of light and 2% carbon dioxide (or 1 g/L of sodium bicarbonate), which is an inorganic carbon source, are added, and the resultant are allowed to react at 30° C. for 4 hours (FIG. 8). Finally, the FAME content in the homogenate is measured by using gas chromatograph (GC).

As described above, the empirical analysis method and a kit for the analysis according to an embodiment of the present invention may largely contribute to microalgae synthetic biological study and microalgae metabolic pathway-related study which is difficult to study through the typical genetic engineering approach, because the following reason: use of a reagent for gene delivery into cells is not required; the biological effect of the effector molecule may be rapidly, and massively analyzed by directly treating the microalgae homogenate with the various effector molecules, which act in cells, beside ligands which bind receptors of cell membrane and deliver signal into cells irrespective of whether the molecules are capable of passing the cell membrane; and multiple factor analysis is easy by combing and treating various effector molecules.

Although the present invention is described with reference to examples depicted in accompanying drawings, the examples are only illustrate, and a person skilled in the art will appreciate that various modifications and other equivalent examples may be embodied from the examples. Thus, the scope of the present invention sough to technical protection should be determined by the technical spirit of accompanying claims.

### INDUSTRIAL APPLICABILITY

The high-performance empirical analysis method of a metabolic pathway in microalgae using a cell-free system according to an embodiment of the present invention may be used as an analytical tool for synthetic biology, and used in improvement of microalgae for biodiesel production.

1. An empirical analysis method of a metabolic pathway using a microalgae cell-free system, comprising:
preparing a microalgae homogenate;
allowing a reaction vessel to contain the microalgae homogenate, and then treating with an effector molecule; and
comparing with a control, which is not treated with the effector molecule, to measure a biological effect of the effector molecule treatment.

2. The empirical analysis method of a metabolic pathway using a microalgae cell-free system of claim 1, wherein, the microalgae is Nostoc sp., Anabaena sp., Crocosphaera sp., Cyanothece sp., Trichormus sp., Richella sp. Calothrix sp., Botryococcus sp., Chlorella sp., Crypthecodinium sp., Arthrospira sp., Cylindrotheca sp., Dunaliella sp., Isochrysis sp., Monallanthus sp., Nannochloris sp., Nannochloropsis sp., Neochloris sp., Nitzschia sp., Phaeodactylum sp., Schizochytrium sp., Tetraselmis sp., or Haematococcus sp microalgae.

3. The empirical analysis method of a metabolic pathway using a microalgae cell-free system of claim 1, wherein the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase.

4. The empirical analysis method of a metabolic pathway using a microalgae cell-free system of claim 3, wherein the effector molecule is a gene construct in which a polynucleotide encoding a certain protein is operatively linked to a promoter operated in microalgae, siRNA, shRNA, miRNA, CRISPRs nucleotide, TALEN nucleotide, or an antisense nucleotide, which may inhibit expression or function of a certain gene inherent in microalgae, an antibody, which inhibits a function of an intracellular protein in microalgae or a functional fragment thereof, a small compound inhibitor, or a substrate analogue which acts as an inhibitor or a substrate of an intracellular enzyme of microalgae.

5. The method of analyzing a function of a microalgae gene of claim 1, wherein the reaction vessel is a single test tube, 6-well, 12-well, 24-well, 48-well, 96-well, 192-well, or 384-well microplate, a microarray, or a microfluidic chamber.

6. A high-performance empirical analysis method of a metabolic pathway using a microalgae cell-free system, comprising:
preparing microalgae homogenate;
allowing a multiwall reaction vessel to contain the microalgae homogenate, and then treating with an effector molecule library;
measuring a biological effect according to the effector molecule treatment; and
comparing with a control, which is not treated with the effector molecule, and selecting an effector molecule which significantly alters the biological effect.

7. The high-performance empirical analysis method of a metabolic pathway using a microalgae cell-free system of claim 6, wherein the microalgae is Nostoc sp., Anabaena sp., Crocosphaera sp., Cyanothece sp., Trichormus sp., Richella sp. Calothrix sp., Botryococcus sp., Chlorella sp., Crypthecodinium sp., Arthrospira sp., Cylindrotheca sp., Dunaliella sp., Isochrysis sp., Monallanthus sp., Nannochloris sp., Nannochloropsis sp., Neochloris sp., Nitzschia sp., Phaeodactylum sp., Schizochytrium sp., Tetraselmis sp., or Haematococcus sp. microalgae.

8. The high-performance empirical analysis method of a metabolic pathway using a microalgae cell-free system of claim 6, wherein the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase.

9. The high-performance empirical analysis method of a metabolic pathway using a microalgae cell-free system of claim 6, wherein the effector molecule library is a gene construct library in which a polynucleotide encoding a certain protein is operatively linked to a promoter operated in microalgae, a gene construct library having a promoter, which is operated by an externally added RNA polymerase, an mRNA library prepared through external transcription, a siRNA, shRNA, miRNA, CRISPRs nucleotide, TALEN nucleotide, or antisense nucleotide library, which may inhibit expression or functions of certain genes inherent in microalgae, a library of an antibody, which inhibits a function of an intracellular protein in microalgae or a functional fragment thereof, or a small compound inhibitor library, a substrate library of an intracellular enzyme in microalgae, a substrate analogue library which acts as an inhibitor, a random mutant or site-directed mutant library of a known gene, or expressed sequence tag (EST) library cloned from metagenome.

10. The high-performance empirical analysis method of a metabolic pathway using a microalgae cell-free system of claim 6, wherein the reaction vessel is a single test tube, 6-well, 12-well, 24-well, 48-well, 96-well, 192-well, or 384-well microplate, a microarray, or a microfluidic chamber

11. A kit for high-performance empirical analysis of a metabolic pathway using a microalgae cell-free system comprising microalgae homogenate.

12. The kit for high-performance empirical analysis of a metabolic pathway using a microalgae cell-free system of claim 11, wherein the microalgae is Nostoc sp., Anabaena sp., Crocosphaera sp., Cyanothece sp., Trichormus sp., Richella sp. Calothrix sp., Botryococcus sp., Chlorella sp., Crypthecodinium sp., Arthrospira sp., Cylindrotheca sp., Dunaliella sp., Isochrysis sp., Monallanthus sp., Nitzschia sp., Phaeodactylum sp., Schizochytrium sp., Tetraselmis sp., or Haematococcus sp. microalgae.

13. The kit for high-performance empirical analysis of a metabolic pathway using a microalgae cell-free system of claim 11, wherein the microalgae homogenate is prepared by homogenizing living microalgae with a bead beater, a homogenizer, a warring blender or a sonicator, and then removing a cell wall component through centrifugation, or degrading a cell wall by using a cell wall degrading enzyme such as cellulase and/or hemicellulase.

14. The kit for high-performance empirical analysis of a metabolic pathway using a microalgae cell-free system of claim 11, further comprising an effector molecule library.

15. The kit for high-performance empirical analysis of a metabolic pathway using a microalgae cell-free system of claim 14, wherein the effector molecule library is a gene construct library in which a polynucleotide encoding a certain protein is operatively linked to a promoter operated in microalgae, an siRNA, shRNA, miRNA, CRISPRs nucleotide, TALEN nucleotide, or antisense nucleotide library, which may inhibit expression or functions of certain genes inherent in microalgae, a library of an antibody, which inhibits a function of an intracellular protein in microalgae or a functional fragment thereof, or a small compound inhibitor, a library of substrates of an intracellular enzyme of microalgae, substrate analogue library which acts as an inhibitor, a random mutant or site-directed mutant library of a known gene, or an expressed sequence tag (EST) library cloned from metagenome.
